(19) United States
(12) Patent Application Publication
(10) Pub. No.: US 2004/0151684 A1
(43) Pub. Date: Aug. 5, 2004

(54) Facial cleansers

(76) Inventors: Noriko Mori, Okayama (JP); Naoto Ogimoto, Okayama (JP); Shukuko Kajinami, Okayama (JP); Shoichi Hamada, Okayama (JP); Keiji Ikeda, Tokyo (JP)

Correspondence Address: BROWDY AND NEIMARK, P.L.L.C., 624 NINTH STREET, NW, SUITE 300, WASHINGTON, DC, 20001-5303, US

(21) Appl. No.: 10/477,147

(22) PCT Filed: May 2, 2002
(86) PCT No.: PCT/JP02/04408

(30) Foreign Application Priority Data
May 7, 2001 (JP) 2001-136348

(51) Int. Cl. A61K 7/06; A61K 7/11

(52) U.S. Cl. 424/70.14; 424/70.13

(57) ABSTRACT

The object of the present invention is to provide a face wash which effectively removes pore stains and aged horny layers of the skin and gives whitening effect but has lesser stimulant feel. The present invention solves the object by providing a face wash comprising a protein-degrading enzyme, lipid-degrading enzyme, trehalose, D-glucose, and ascorbic acid and/or a derivative thereof as effective ingredients.

### TECHNICAL FIELD

The present invention relates to a novel face wash, more particularly, to a face wash which comprises an enzyme, saccharide, and ascorbic acid and/or derivative thereof as effective ingredients.

### BACKGROUND ART

Face washes are mainly used for removing excessively secreted sebum, stains and make-up cosmetics on the skin surface, and are useful to recover and promote the metabolism of the skin tissue and to keep the skin clean and healthy conditions. There are three functions required in face washes; first, lesser stimulant feel; second, removal of excessively secreted sebum on the skin surface, stains in the pores, and aged horny layers; third, recovery and promotion of the metabolism of the skin tissue and function of keeping the skin clean and in healthy conditions. There have been tried to further incorporate various ingredients for balancing, sterilizing and moisturizing the skin into face washes to impart them additional functions. However, conventional face washes are insufficient in the above three basic functions and would not fulfil additional functions.

Under the above circumstances, it has been required a novel face wash which has the above three basic functions required in them at a higher level and imparts a satisfactory elasticity, gloss, and transparency to the skin to make it beautiful white skin.

### DISCLOSURE OF INVENTION

In view of the prior arts, the object of the present invention is to provide a face wash which has the above three basic functions required thereto at a higher level and imparts a satisfactory elasticity, gloss, and, as an additional function, transparency to the skin to make it beautiful white skin.

The present inventors eagerly studied to solve the above object. As a result, they found that a face wash comprising a protein-degrading enzyme, lipid-degrading enzyme, trehalose, D-glucose, and ascorbic acid and/or derivative thereof as effective ingredients solves the aforementioned object. Thus, they accomplished the present invention.

### BEST MODE FOR CARRYING OUT THE INVENTION

Explaining the face wash of the present invention, the protein-degrading enzymes, used in the present invention and mainly added with the aim of efficiently removing aged horny layers from the skin, are not specifically restricted as long as they fulfil the aim and are cosmetically acceptable. Concrete examples of such are those from microorganisms of the genera such as Bacillus and Actinomyces, in a cosmetically acceptable form. Papain can be also used as such. Varying the form and use of the face wash of the present invention, it preferably contains any of proteases, usually, in an amount of 0.01 unit or more, desirably, 0.1 to 10,000 units, more desirably, 0.1 to 1,000 units, most desirably, 0.1 to 100 units per one gram of the product.

The lipid-degrading enzymes, used in the present invention and mainly added with the aim of efficiently removing excessively secreted sebum on the skin and stains in the pores, are not specifically restricted as long as they fulfil the aim and are cosmetically acceptable. Concrete examples of such are those from microorganisms such as yeasts and those of the genus such as Lizobus, in a cosmetically acceptable form. Varying the form and use of the face wash of the present invention, it preferably contains any of lipases, usually, in an amount of 0.01 unit or more, desirably, 0.1 to 100,000 units, more desirably, 1 to 10,000 units, most desirably, 10 to 1,000 units per one gram of the face wash.

The trehalose used in the present invention is a non-reducing succharide (a disaccharide) composed of D-glucoses and widely present in natural resources such as microorganisms, mushrooms, and insects. Recently, it is enzymatically produced on an industrial scale at a lesser cost. Since trehalose protects epidermal cells in the skin tissue against the actions of the aforesaid protein-degrading enzymes and lipid-degrading enzymes and acts as an energy source to activate the cells, it is added to impart elasticity, gloss and humectancy or moisture to the skin after face washing in preparing the face wash of the present invention. Any trehalose can be used as long as it fulfils the above aim and is cosmetically acceptable. Examples of such trehaloses are those obtained, for example, by the methods described in Japanese Patent Kokai Nos. 356,600/94, 143,876/95, 213,283/95, 170,977/95, 322,880/95, 322,883/95, 298,887/95, 298,880/95, 263/96, 66,188/96, 66,187/96, 73,504/96, 73,506/96, 9,986/97, 84,586/96, 336,388/96, 149,980/96, and 217,784/96. In the present invention, those safe and inexpensive trehaloses obtained by the above methods can be preferably used. Any trehalose in the form of a crystalline dihydrate, crystalline anhydrate, amorphous, syrup, powder or granule can be used in the present invention. The purity of those trehaloses should not be restricted and it may be from a relatively low purity to the highest purity as long as it does not hinder the object of the present invention. The desired purity of the trehalose is, usually, 70%(w/w) or more, preferably 80%(w/w) or more, and more preferably 90%(w/w) or more. Varying the form and the use of the face wash of the present invention, the percentage of the trehalose in the face wash is usually 5 to 99%(w/w), preferably 10 to 80%(w/w), and more preferably 30 to 70%(w/w) on a dry solid basis (hereinafter designated as “d.s.b.”). In the present invention, neotrehalose (α,β-trehalose) and/or isotrehalose (β,β-trehalose), known as isomers of trehalose, can be used with the trehalose as long as they do not hinder the aim of the use of the trehalose. And, one or more non-reducing trisaccharides or larger having a trehalose structure intramolecularly or at the ends of their molecules, described in the above Japanese Patent Applications, can be used arbitrarily.

Since, like the above-mentioned trehalose, D-glucose protects the epidermal cells in the skin tissue against the actions of the aforesaid protein-degrading enzymes and lipid-degrading enzymes and acts as an energy source to activate the cells, it is added to the face wash of the present invention as an excipient. Any D-glucose can be used as long as it fulfils the above aim and is cosmetically acceptable. The D-glucose usable in the present invention includes those in the form of a crystalline monohydrate, crystalline anhydrate, amorphous, syrup, powder, or granule. The purity of D-glucose in such a form should not be restricted and it may be from a relatively low purity to the highest purity as long as it does not hinder the object of the present invention. The desired purity of the D-glucose is, usually 70%(w/w) or more, preferably 80%(w/w) or more, and more preferably 90%(w/w) or more. Varying the form and the use of the face wash of the present invention, the percentage of the D-glucose is usually 1 to 99%(w/w), preferably 10 to 80%(w/w), and more preferably 30 to 70%(w/w), d.s.b.

Ascorbic acid or derivatives thereof as referred to as in the present invention mean compounds which release L-ascorbic acid when it is placed in an aqueous medium or administered to a living body, and is cosmetically acceptable. Concretely, L-ascorbic acid, α-glucosyl-L-ascorbic acid having a structure that L-ascorbic acid binds one or more α-D-glucoses at the alcohol base of C-2 of L-ascorbic acid described in Japanese Patent Kokai No. 135,992/91, and a-glycosyl-L-ascorbic acid (these L-ascorbic acid derivatives are simply called “α-glycosyl-L-ascorbic acids”), acylated derivatives of α-glycosyl-L-ascorbic acid described in Japanese Patent Kokai No. 286,497/99, α-glycosyl-L-ascorbic acid having a structure that L-ascorbic acid binds one or more α-D-glucoses at the alcohol base of C-5 of L-ascorbic acid described in Japanese Patent Kokai No. 112,594/93, and metal salts of α-glycosyl-L-ascorbic acid described in Japanese Patent Kokai No. 220,081/94 can be mentioned. The above L-ascorbic acid or derivatives thereof are added to the face wash of the present invention with the aim of moisturizing and whitening the skin. Any L-ascorbic acid or derivatives in the form of a crystalline dihydrate, crystalline anhydrate, amorphous, syrup, powder, or granule can be used in the present invention. The purity of such should not be restricted and may be from a relative low purity to the highest purity as long as it does not hinder the object of the present invention. The desired purity of L-ascorbic acid or derivatives thereof is, usually 70%(w/w) or more, preferably 80%(w/w) or more, and more preferably 90%(w/w) or more. Varying the form and the use of the face wash of the present invention, the percentage of L-ascorbic acid or derivatives thereof in the face wash is usually 0.001%(w/w) or more, preferably 0.01 to 50%(w/w), preferably 0.01 to 30%(w/w), and more preferably 0.1 to 20%(w/w), d.s.b.

Before completion of the preparation of the face wash of the present invention, it can be combined with one or two more ingredients selected from the group consisting of saccharides other than trehalose and D-glucose, antimicrobials, sterilizers, disinfectants, antifungals, antiseptics, antioxidants, moisturizers, antiinflammatory agents, pH adjusters, osmotic adjusters, cell activators, gums, excipients, binders, preservatives, stabilizers, astringents, lipid peroxide producing inhibitors, vitamins other than ascorbic acid, amino acids, minerals, photosensitive dyes, alcohols, water, aqueous solvents, deep-sea water, sea water, oily solvents, UV-absorbers, whitening agents, detergents, animal extracts, plant extracts, seaweed extracts, proteins, coloring agents and flavors, which are all in a cosmetically acceptable form. These ingredients can be added in the face wash of the present invention as long as they do not hinder the aim of its use, and they are partly shown as follows:

Examples of the saccharides other than trehalose and D-glucose are monosacchrides such as D-mannose, D-galactose, D-allose, D-altrose, D-talose, D-tagatose, D-psicose, D-idose, D-arabinose, D-sorbose, D-ribose, D-xylose, D-lyxose, D-lactose, and D-fructose; disaccharides such as maltose, isomaltose, turanose, sucrose, lactose, turanose, sophorose, cellobiose, gentibiose, melibiose; oligosaccharide larger than disaccharides; polysaccharides; and derivatives thereof including their sugar alcohols, which are all in a cosmetically acceptable form,

Examples of the whitening agents are koji acid or derivatives thereof, lactic acid or derivatives thereof, p-aminobenzoic acid derivatives, salicylic acid derivatives, anthranilic acid derivatives, coumarin derivatives, amino acid compounds, benzotriazole derivatives, tetrazole derivatives, imidazoline derivatives, pyrimidine derivatives, dioxane derivatives, camphor derivatives, furan derivatives, pyrone derivatives, nucleotide derivatives, nicotinic acid derivatives, oxybenzone, benzophenone, arbutin, shikonin, baicalin, baicalein, berberine, melanogenesis inhibitors, tyrosinase generating inhibitor, and tyrosinase activity inhibitor.

Examples of the antiinflammatory agents are allantoin, allantoin derivatives, ichthammol, indometacin, kaolin, salicylic acid, sodium salicylate, methyl salicylate, acetyl salicylate, diphenhydramine hydrochloride, d-camphor, dl-camphor, hydrocortisone, guaiazulene, chamazulene, chlorpheniramine maleate, glycyrrhizic acid or salt thereof, α-glucosylglycyrrhizin, α-glycosylglycyrrhizin, Lithospermum erythrorhizone root extract, and rose extract.

Examples of the photosensitive dyes having cell activating effects are KANKOH-SO 101 (product name “PLATONIN”), KANKOH-SO 201, (product name “PIONIN”), KANKOH-SO 301 (product name “TAKANAL”), KANKOH-SO 401 (product name “LUMINEX”), and KANKOH-SO NK-143 (product name “PLALUMIN”) (commercialized by Hayashibara Biochemical Laboratories, Inc., Okayama, Japan).

Examples of the gums are natural organic polymers such as gum arabic, tarmarind gum, karaya gum, tragacanth gum, quince seed gum, agar, casein, dextran, dextrin, cellulose, gelatin, pectin, sodium pectate, starch, sodium alginate, carrageenan, xanthan gum, and pullulan, synthetic polymers such as polyvinylpyrrolidone, polyvinylpyrrolidone-vinylacetate copolymer, polyethyleneglycol, polyvinylalcohol, pullulan derivative, polyvinylmethylether, sodium polyacrylate, carboxyvinylpolymer, polyethyleneimine, methylcellulose, ethylcellulose, acethylcellulose, nitrocellulose, vinyl acetate resin, crystalline cellulose, hydroxyethylcellulose, hydroxypropylcellulose, carboxymethylcellulose, sodium carboxymethylcellulose, hydroxyp ropylmethylcellulose, hydroxypropylmethylcellulose phthalate, cellulose acetate, phthalate, cellulose propyonate phthalate, cellulose acetate malate, polyvinylalcohol phthalate, styrene-acrylic acid copolymer, methylacrylate-methacrylate copolymer, acrylic acud copolymer with acetate, vinylpyridine or alkylvinyl pyridine, inorganic compounds such as montmorillonite, kaolin, silica hydrate, aluminum magnesium silicate, calcium carbonate, and hectorite.

The process for producing the face wash of the present invention includes any method as long as it incorporates protein-degrading enzymes, lipid-degrading enzymes, trehalose, D-glucose, and ascorbic acid or derivatives thereof as effective ingredients into the final products in an amount of the above mentioned percentages before completion of its processing. The above effective ingredients can be mixed and granulated manually or mechanically; or mixed, dispersed, suspended or dissolved homogeneously in one or more appropriate solvents such as water, hydrophilic solvents, and hydrophobic solvents. The method for mixing, dispersing, suspending or dissolving the above effective ingredients is not specially restricted. The temperature for mixing the above ingredients is usually 0° C. or more, preferably 5 to 100° C., more preferably 10 to 70° C., and most preferably 10 to 50° C. The face wash of the present invention can be produced by freeze-drying a solution containing the effective ingredients used in the present invention at a low temperature of below 0° C. The above process is preferably manipulated with an antioxidant(s), under a light shielded, or unoxic condition for inhibiting the inactivation of ascorbic acid and derivatives thereof. The face wash of the present invention is preferably sterilized by conventional methods such as filtrating, heating, pressurizing, radiating, UV-irradiating, ozone-treating, and drug-treating to avoid microbial contamination. The face wash of the present invention optionally contains drugs such as antimicrobials, sterilizers, disinfectants, antifungals, and antiseptics for inhibiting microbial contamination during preservation. However, the use of such drugs should be minimized when used for delicate skins. When the face wash of the present invention is in the form of a paste, suspension, foam, or solution, it should be adjusted usually to a pH of 4.5 to 8, preferably 5 to 7.5, and more preferably 5 to 7.2.

Examples of the form of the face wash of the present invention include those in the form of a solid such as a powder, granule, gel, paste, foam, or liquid and can be processed by attaching or soaking to absorbent materials such as a cotton, cloth, paper, sponge, and nonwoven cloth. The face wash of the present invention in any of the above forms can be filled or packed into an appropriate container or potion-type container for a final product.

The use of the face wash of the present invention is explained below: When in the form of a solid such as a powder or granule, varying due to the amount of the effective ingredients contained in the face wash, it is dissolved in a mount of, usually, 0.01 g or more, preferably, 0.1 to 100 g, more preferably, 0.5 to 10 g, and most preferably, 0.5 to 5 g in an appropriate amount water, lukewarm water, or warm water before use. When it is dissolved in a small amount of water, lukewarm water, or warm water, it can be first dissolved therein on a user's palm. Next, the dissolved face wash is applied gently and uniformly unto a part or the whole of the user's face skin and allowed to stand for about 0.5 to five minutes, preferably, about one to two minutes, and then carefully rinsed out with water, lukewarm water, or warm water.

When the face wash is in the form of a solid or liquid such as a gel, paste, foam, or liquid, it is taken on a user's hand in an amount of, usually 0.1 to 5 g, preferably 0.5 to 3 g, and more preferably 0.5 to 1 g, alone or after dissolved or suspended in an appropriate amount of water, lukewarm water, or warm water. Next, the resulting mixture is applied gently and uniformly unto a part or the whole of the user's face skin and allowed to stand for about 0.5 to five minutes, preferably, about one to three minutes, and then carefully rinsed out with water, lukewarm water, or warm water. When the face wash is allowed to stand on the face skin, a steamed, warmed towel is applied to the user's face skin to promote the actions of protein-degrading enzymes and lipid-degrading enzymes. The face wash can be applied onto a part of the user's face skin, particularly, caring positions such as soiled pores, dull skins, stained skins, and freckled skins. In use, the face wash is not specifically restricted to specific daily frequency but effectively used usually one to five times per day, preferably, one to three times per day. After face washing, any treatments, according to user's life style, they are allowed to make up with lotions, milky lotions, creams, oils, face packs, or make-up cosmetics without any restriction.

The following experiments explain the face wash of the present invention in more detail:

Experiment: Face Washing Test

A panel test was conducted using the face wash of the present invention with the formulation A and, as controls, those prepared with the formulation B (B-1 to B-6) in Table 1. Panels for the panel test, composed of 42 healthy adults (21 males and 21 females) aged 20 to 50, were divided into seven groups of G1 to G7 groups, consisting of six panels (3 males and 3 females) each. Before the panel test or the face washing test, all the panels were checked about the conditions of the face skin by an esthetician. The panels of G1 group with the face wash of the formulation A of the present invention and those of G2 to G7 groups with the face washes as controls with the formulations B-1 to B-6, respectively, were allowed to wash their faces once every morning for successive 30 days. The panels were instructed to wash their faces in such a manner of taking 2 ml of their assigned face washes described in the formulations A and B-1 to B-6, applying them on the whole of their face skins gently and uniformly, allowing the face washes to stand for two minutes, and rinsing with lukewarm water (about 30 to about 35° C.). After face washing, the panels were allowed to make up freely on their own way and to have their own lives.

TABLE 1

| | Formu- | | | | | | |
|---|---|---|---|---|---|---|---|
| | lation | | | | | | |
| | A | | | | | | |
| | (the | | | | | | |
| | present | Formulation B (control) | | | | | |
| | invention) | B-1 | B-2 | B-3 | B-4 | B-5 | B-6 |
| Trehalose (g) | 7.6 | 7.6 | 7.6 | 7.6 | 7.6 | — | — |
| D-Glucose (g) | 6 | 6 | 6 | 6 | 6 | — | — |
| Protease¹⁾ | 0.15 | — | 0.15 | — | 0.15 | 0.15 | 0.15 |
| (Protein- | | | | | | | |
| degrading | | | | | | | |
| enzyme) (g) | | | | | | | |
| Lipase²⁾ | 0.03 | 0.03 | — | — | 0.03 | 0.03 | 0.03 |
| (Lipid-degrading | | | | | | | |
| enzyme) (g) | | | | | | | |
| 2-O-α-D- | 0.15 | 0.15 | 0.15 | 0.15 | — | 0.15 | — |
| Glucopyranosyl- | | | | | | | |
| L-ascorbic acid | | | | | | | |
| (g) | | | | | | | |
| Sodium | 1.05 | 1.05 | 1.05 | 1.05 | 1.05 | 1.05 | 1.05 |
| carboxymethyl- | | | | | | | |
| cellurose | | | | | | | |

| | |
|---|---|
| pH Adjuster | Appropriate amount to adjust pH to 7.0 |
| Purified water | Appropriate amount to give the total volume of 100 ml |

Note:
¹⁾In the formulations A and B, their protease activities were adjusted to 0.0075 unit/ml. The protease used in this experiment was prepared by diluting “BIOPRASE CONC” (150,000 units/g, commercialized by Nagase Chemtex Corporation, Osaka, Japan) with trehalose to give a concentration of 5 units/g.
²⁾In the formulations A and B, their lipase activities were adjusted to 0.3 unit/ml. The lipase used in this experiment was prepared by diluting “LIPASE MY-30” (30,000 units/g, commercialized by Meito Sangyo Co., Ltd., Aichi, Japan) with trehalose to give a concentration of 1,000 units/g.

After the face washing test for 30 days, all the panels were allowed to evaluate themselves about the face washes of the formulation A of the present invention and of the formulation B (B-1 to B-6) as controls on the three questionaire items, i.e., stimulant feel, moisture feel, and tightening feel of the skin after face washing. While, the esthetician evaluated the conditions of the panels' face skins in comparison with those before the face washing test. Four items checked by the esthetician were the effects of removing pore stains, removing aged horny layers, whitening, and degree of skin inflammation. The items of moisture feel and tightening feel of the skin as questionnaire items, and those of the effects on removing pore stains, removing aged horny layers, and skin whitening checked by the esthetician were evaluated in such a manner of scoring “very good”, 5 points; “good”, 4 points; “passable”, 3 points; “rather no good”, 2 points; and “no good”, 1 point. The stimulant feel as one of the questionnaire items was evaluated by the answers from the panels, and the degree of skin inflammation was checked by the esthetician. The results are summarized in Table 2. In this table, the values in “X” column are the total points scored by the panels for each item, and those in “Y” column are the total points scored by the esthetician for the panels for each group.

TABLE 2

| | | Formu- | | | | | | |
|---|---|---|---|---|---|---|---|---|
| | | lation A | | | | | | |
| | | (the | | | | | | |
| | | present | Formulation B (control) | | | | | |
| | | invention) | B-1 | B-2 | B-3 | B-4 | B-5 | B-6 |
| Items | | (G1) | (G2) | (G3) | (G4) | (G5) | (G6) | (G7) |
| X | Stimulant | None | None | None | None | None | None | None |
| | feel | | | | | | | |
| | Moisture feel | 27 | 27 | 27 | 26 | 25 | 6 | 6 |
| | Tightening | 28 | 27 | 27 | 27 | 26 | 7 | 6 |
| | feel | | | | | | | |
| Y | Effect of | 29 | 10 | 23 | 6 | 28 | 27 | 27 |
| | removing | | | | | | | |
| | aged | | | | | | | |
| | horny layer | | | | | | | |
| | Effect of | 29 | 22 | 7 | 6 | 27 | 27 | 26 |
| | removing | | | | | | | |
| | pore | | | | | | | |
| | stain | | | | | | | |
| | Effect of | 27 | 20 | 21 | 21 | 7 | 19 | 6 |
| | whitening | | | | | | | |
| | the skin | | | | | | | |
| | Inflam- | None | None | None | None | None | None | None |
| | matory | | | | | | | |
| | to the skin | | | | | | | |

Note:
In the table, the values in “X” column are the results of the answers from the panels, and those in “Y” column are of the evaluations by the esthetician.
Each value in the table means the total points of the panels for each group.

As evident from the results in Table 2, it was revealed that the protein-degrading enzyme, incorporated in the face wash of the present invention as an effective ingredient, effectively removed aged horny layer; and the lipid-degrading enzyme effectively removed pore stain, indicating that the combination use of the enzymes was remarkably more effective than their sole use. It was also revealed that trehalose, D-glucose, and 2-O-D-α-glucopyranosyl-L-ascorbic acid as an ascorbic acid derivative prevent the skin from roughing induced by face washing and impart moisture feel (moisturizing effect) to the skin after face washing, effectively reduce tightening feel after face washing, impart elasticity and gloss to the skin, and whiten the skin. It was also revealed that all the ingredients in the face wash of the present invention are free from causing inflammation of the skin, and the combination use of a protein-degrading enzyme, lipid-degrading enzyme, trehalose, D-glucose, and L-ascorbic acid and/or a derivative(s) thereof in the face wash of the present invention, as effective ingredients, effectively remove pore stain and aged horny layer and whiten the skin in a level exceeding that of their sole use. Similar results will be obtained from the face wash with the formulation A, which comprises another ascorbic acid derivative(s), usable in the present invention, instead of 2-O-D-α-glucopyranosyl-L-ascorbic acid.

Furthermore, among the panels with the face wash of the formulation A of the present invention, five out of six panels who washed their face with their daily used face washes were asked about the difference between the face wash with the formulation A of the present invention and their daily used ones. As a result, all the five panels answered that the face wash of the present invention was more effective than their daily used face washes in removing pore stain and imparting transparency (whitening effect) to the skin.

The following examples explain the face wash of the present invention in more detail:

### EXAMPLE 1

### Powder-Type Face Wash

Fifty-one parts by weight of “COSMETIC TREHALOSE”, a product name of a crystalline trehalose dihydrate trehalose powder, commercialized by Hayashibara Shoji Co., Ltd., Okayama, Japan, (hereinafter simply called “Trehalose” throughout the following Examples), 40 parts by weight of D-glucose, seven parts by weight of sodium carboxymethylcellulose, one part by weight of “BIOECOLIA”, a product name of a maltose-sucrose condensate commercialized by Nikko Chemicals Co., Ltd., Tokyo, Japan, one part by weight of “AA2G”, a product name of 2-O-α-D-glucosyl-L-ascorbic acid with a purity of 98.0%(w/w) or more commercialized by Hayashibara Shoji Co., Ltd., Okayama, Japan, 0.1 part by weight of allantoin commercialized by Kawaken Fine Chemicals Co., Ltd., Tokyo, Japan, 0.2 part by weight of trehalose containing 1,000 units/g of “LIPASE MY-30”, a product name of lipase (30,000 units/g) commercialized by Meito Sangyo Co., Ltd., Aichi, Japan, 0.002 part by weight of “KANKOH-SO 401” commercialized by Hayashibara Biochemical Laboratories, Co. Inc., Okayama, Japan, and one part by weight of trehalose containing five units/g of “BIOPRASE CONC”, a product name of a protease commercialized by Nagase Chemtex Corporation, Osaka, Japan, were mixed and passed through a 26-mesh sieve three times to homogeniety to obtain a powdery product. 0.3 g of the powdery product was injected into a package to obtain a face wash in a powder form of the present invention.

In a practical use, the present product is preferably used by the steps of dissolving one package of the product in 2 ml of water or lukewarm water, applying the resulting solution to the face skin gently and uniformly, allowing the skin with the application to stand for two or three minutes, and rinsing the skin with water or lukewarm water to carefully remove the applied product. The product has satisfactory handiness, no stimulant feel in use and no tightening feel after use. The daily use of the product will remove excess sebum and aged horny layer on the surface of the face skin and pore stain effectively to promote skin metabolism. As a result, it would impart a satisfactory elasticity, gloss, and transparently beautiful white to the skin.

### EXAMPLE 2

### Aerosol-Type Face Wash

Six hundred and sixty-seven parts by weight of purified water, 60 parts by weight of trehalose, 20 parts by weight of D-glucose, 20 parts by weight of maltose, five parts by weight of pullulan, one part by weight of “BIOECOLIA”, a product name of a maltose-sucrose condensate commercialized by Nikko Chemicals Co., Ltd., Tokyo, Japan, 10 parts by weight of “AA2G”, a product name of a 2-O-α-D-glucosyl-L-ascorbic acid powder with a purity of 98.0%(w/w) or more commercialized by Hayashibara Shoji Co., Ltd., Okayama, Japan, 0.2 part by weight of allantoin commercialized by Kawaken Fine Chemicals Co., Ltd., Tokyo, Japan, 0.4 part by weight of trehalose containing 1,000 units/g of “LIPASE MY-30”, a product name of lipase (30,000 units/g) commercialized by Meito Sangyo Co., Ltd., Aichi, Japan, 0.002 part by weight of “KANKO-SO 301” commercialized by Hayashibara Biochemical Laboratories, Inc., Okayama, Japan, and two parts by weight of trehalose containing five units/g of “BIOPRASE CONC”, a product name of protease (150,000 units/g) commercialized by Nagase Chemtex Corporation, Osaka, Japan, and 37 parts by weight of polyethylene glycol (1500) were homogenized by a mixer. To the resultant solution was added a solution containing three parts by weight of heat-melted P.O.E (20) octyldodecylether and 30 parts by weight of heat-melted polyethylene glycol (1500), followed by dissolution and filtration. The resultant filtrate was injected into an appropriate container which was then injected with 3.5 parts by weight of nitrogen gas as a propellant gas to obtain an aerosol-type face wash of the present invention.

In a practical use, the product is preferably used by the steps of taking an appropriate amount thereof on the palm, applying it with water or lukewarm water to the face skin gently and uniformly, allowing the resultant to stand thereupon for two to three minutes, and rinsing the face with water or lukewarm water to carefully remove the applied product. The product has satisfactory handiness, no stimulant feel in use, and no tightening feel after use. The daily use of the product for face washing will remove excess sebum and aged horny layers on the surface of the face skin and pore stains effectively to promote the skin metabolism. As a result, it would impart a satisfactory elasticity, gloss, and transparently beautiful white to the skin.

### EXAMPLE 3

### Lotion-Type Face Wash

Five hundred parts by weight of purified water, 100 parts by weight of trehalose, 10 parts by weight of D-glucose, one part by weight of dextrin, one part by weight of pectin, one part by weight of fructose, one part by weight of “AA2G”, a product name of a 2-O-α-D-glucosyl-L-ascorbic acid powder with a purity of 98.0%(w/w) or more commercialized by Hayashibara Shoji Co., Ltd., Okayama, Japan, one part by weight of L-ascorbic acid, 0.2 part by weight of salicylic acid, two parts by weight of trehalose containing 1,000 units/g “LIPASE MY-30”, a product name of lipase (30.000 units/g) commercialized by Meito Sangyo Co., Ltd., Aichi, Japan, 0.2 part by weight of “KANKOH-SO 401” commercialized by Hayashibara Biochemical Laboratories, Inc., Okayama, Japan, and 10 parts by weight of trehalose containing five units/g of “BIOPRASE CONC”, a product name of protease (150,000 units/g) commercialized by Nagase Chemtex Corporation, Osaka, Japan, 0.2 part by weight of “KANKOH-SO 201” commercialized by Hayashibara Biochemical Laboratories, Inc., Okayama, Japan, 0.1 part by weight of collagen, and 30 parts by weight of paraben were mixed to a obtain lotion-type face wash of the present invention.

In a practical use, the product is preferably used by the steps of taking 1 to 5 ml of the product on the palm, applying it to the face skin gently and uniformly, allowing the application to stand thereupon the skin for two to three minutes, and rinsing the face with water or lukewarm water to carefully remove the applied product. The product has satisfactory handiness, no stimulant feel in use, and no tightening feel after use. The daily use of the product for face wash will remove excess sebum and aged horny layers on the surface of the face skin and pore stains effectively to promote the skin metabolism. As a result, it would impart a satisfactory elasticity, gloss, and transparently beautiful white to the skin.

### EXAMPLE 4

### Cream-Type Face Wash

Forty parts by weight of purified water, 20 parts by weight of trehalose, 5 parts by weight of D-glucose, five parts by weight of maltose, five parts by weight of maltitol, five parts by weight of lactosucrose, five parts by weight of “AA2G”, a product name a of 2-O-α-D-glucosyl-L-ascorbic acid powder with a purity of 98.0%(w/w) or more commercialized by Hayashibara Shoji Co., Ltd., Okayama, Japan, 0.5 part by weight of trehalose containing 1,000 units/g “LIPASE MY-30”, a product name of lipase (30.000 units/g) commercialized by Meito Sangyo Co., Ltd., Aichi, Japan, two parts by weight of trehalose containing five units/g of “BIOPRASE CONC”, a product name of protease (150,000 units/g) commercialized by Nagase Chemtex Corporation, Osaka, Japan, 20 parts by weight of myristic acid, five parts by weight of stearic acid, and eight parts by weight of propylene glycol, five parts by weight of potassium hydroxide, five parts by weight of coconut fatty acid diethanolamide, 0.01 part by weight of α-glycosylhesperidin, and appropriate amounts of a lemon fragrant and a preservative were mixed to obtain a cream-type face wash of the present invention.

In a practical use, the product is preferably used by the steps of taking 1 to 5 g of the product on the palm, applying it to the face skin gently and uniformly, allowing the application to stand thereupon for two to three minutes, and rinsing the face with water or lukewarm water to carefully remove the applied product. The product has satisfactory handiness, no stimulant feel in use, and no tightening feel after use. The daily use of the product for face wash will remove excess sebum and aged horny layers on the surface of the face skin and pore stains effectively to promote the skin metabolism. As a result, it would impart a satisfactory elasticity, gloss, and transparently beautiful white to the skin.

### EXAMPLE 5

### Cleansing Oil-Type Face Wash

Thirty parts by weight of purified water, 20 parts by weight of trehalose, 20 parts by weight of D-glucose, 10 parts by weight of lactose, six parts by weight of “AA2G”, a product name of a 2-O-α-D-glucosyl-L-ascorbic acid powder with a purity of 98.0%(w/w) or more commercialized by Hayashibara Shoji Co., Ltd., Okayama, Japan, 0.2 part by weight of trehalose containing 1,000 units/g “LIPASE MY-30”, a product name of lipase (30.000 units/g) commercialized by Meito Sangyo Co., Ltd., Aichi, Japan, one part by weight of trehalose containing five units/g of “BIOPRASE CONC.”, a product name of protease (150,000 units/g) commercialized by Nagase Chemtex Corporation, Osaka, Japan, 10 parts by weight of pullulan, 100 parts by weight of liquid paraffin, 20 parts by weight of beeswax, one part by weight of sorbitan sesquioleate, 0.01 part by weight of α-glycosylnarindin, three parts by weight of lanolin, three parts by weight of a sulfur powder, and appropriate amount of a lemon fragrant and a preservative were mixed to obtain a cleansing oil-type face wash of the present invention.

In a practical use, the product is preferably used by the steps of taking 1 to 5 g of the product on the palm, applying it to the face skin gently and uniformly, allowing the application to stand thereupon for two to three minutes, and rinsing the face with water or lukewarm water to carefully remove the applied product. The product has satisfactory handiness, no stimulant feel in use, and no tightening feel after use. The daily use of the product for face washing will remove excess sebum and aged horny layers on the surface of the face skin and pore stains effectively to promote the skin metabolism. As a result, it would impart a satisfactory elasticity, gloss, and transparently beautiful white to the skin.

### EXAMPLE 6

### Portion-Type Face Wash

The face washes in Examples 3 to 5 were allowed to hold in rectangular cotton pieces (3×5 cm) and optionally removed excessive amounts of the face washes by pressing them slightly from their upper side, and then, each resultant cotton pieces were sealed in an appropriate package to obtain three kinds of portion-type face washes.

The products are preferably used by the steps of taking them from the packages, applying them to the face skin in such a manner of ether out pressing them to caring parts such as soiled pores, dull skins, stained skins, and freckled skins, or wiping the skins; and allowing the resultant to stand on the skin for two to three minutes, and rinsing the face skin with water or lukewarm water to carefully remove the applied product. They have satisfactory handiness and portability for use in traveling.

### INDUSTRIAL APPLICABILITY

As described above, the face wash of the present invention, which comprises a protein-degrading enzyme, lipid-degrading enzyme, trehalose, D-glucose, and ascorbic acid and/or a derivative thereof as effective ingredients, effectively removes excessively secreted sebum, stains, pore stains, and aged horny layers and recovers and promotes the skin metabolism to impart satisfactory elasticity and gloss to the skin and make a dull skin to a transparently beautiful white skin. In addition, the face wash has substantially no stimulant feel and it would be an ideal face wash.

Thus, the present invention will give a great influence on the art.

1. A face wash which comprises as effective ingredients a protein-degrading enzyme, lipid-degrading enzyme, trehalose, D-glucose, and ascorbic acid and/or a derivative thereof.

2. The face wash of claim 1, wherein said protein-degrading enzyme is a protease.

3. The face wash of claim 1, wherein said lipid-degrading enzyme is a lipase.

4. The face wash of any one of claims 1 to 3, wherein said ascorbic acid and/or said derivative thereof are one or more members selected from the group consisting of L-ascorbic acid, α-glucosyl-L-ascorbic acid, α-glycosyl-L-ascorbic acid, and derivatives thereof.

5. The face wash of any one of claims 1 to 4, which further comprises one or more ingredients selected from the group consisting of saccharides other than trehalose and D-glucose, antimicrobials, sterilizers, disinfectants, antifungals, antiseptics, antioxidants, moisturizers, antiinflammatories, pH adjusters, osmotic adjusters, cell activators, gums, excipients, binders, preservatives, stabilizers, astringents, lipid peroxide production inhibitors, vitamins other than ascorbic acid, amino acids, minerals, photosensitisers, alcohols, water, aqueous components, deep-sea water, sea water, oily components, UV-absorbers, whitening agents, detergents, animal extracts, plant extracts, seaweed extracts, proteins, colorants and flavors,

6. The face wash of claim 5, wherein said saccharides other than trehalose and D-glucose are one or more members selected from the group consisting of monosaccharides, oligosaccharides, polysaccharides, and sugar alcohols thereof.

7. The face wash of claim 5, wherein said vitamins other than ascorbic acid are one or more members selected from the group consisting of vitamins classified as vitamin A, vitamin B, vitamin D, vitamin E, vitamin K, and derivatives thereof; quercetin; α-glucoryl-quercetin; α-glycosyl-quercetin; narindin; α-glucosyl-narindin; α-glycosyl-narindin; hesperidin; α-glucoryl-hesperidin; and α-glycosyl-hesperidin.

8. The face wash of any one of claims 1 to 7, which is in the form of a powder, granule, gel, paste, foam, or liquid.

9. The face wash of any one of claims 1 to 8, which is in the form of being attached or soaked to a moisture-absorbable material.

10. The face wash of any one of claims 1 to 9, which is of a potion-type.
